(19) United States
(12) Patent Application Publication
(10) Pub. No.: US 2022/0389458 A1
(43) Pub. Date: Dec. 8, 2022

(54) LOW VOLUME TRANSFECTION

(71) Applicant: DSM IP Assets B.V., Heerlen (NL)

(72) Inventors: Maarten Dirk VERHOEVEN, Echt (NL); Johannes Andries ROUBOS, Echt (NL); Noel Nicolaas Maria Elisabeth VAN PEIJ, Echt (NL)

(21) Appl. No.: 17/771,333

(22) PCT Filed: Nov. 2, 2020
(86) PCT No.: PCT/EP2020/080625

§ 371 Date: Apr. 22, 2022

(30) Foreign Application Priority Data
Nov. 4, 2019 (EP) 19206817.9

(51) Int. Cl. C12N 15/87; C12N 15/80

(52) U.S. Cl. CPC C12N 15/87; C12N 15/80

(57) ABSTRACT

The invention relates to an improved method for the transfection of fungal cells.

### Field of the invention

The invention relates to an improved method for the transfection of fungal cells.

### BACKGROUND OF THE INVENTION

In the last decade, technological developments in genomics have rapidly accelerated and many new techniques, such as CRISPR-Cas-mediated genome editing, have become available. Improvement of methods for transfecting the compounds required for such genomics techniques into fungal cells have lagged behind the rapid development of the genomics techniques. Accordingly, there is a need for improved transfection techniques, e.g. to be able to transfect large numbers of controlled transfections of fungal cells in parallel in a more efficient way. The invention addresses above described need and provides such technique.

### SUMMARY OF THE INVENTION

Provided is a method for transfecting a compound into fungal cells, preferably filamentous fungal cells, comprising:
(a) providing a container,
(b) dispensing into the container at least:
   an amount of an aqueous solution comprising at least the compound to be transfected,
   an amount of fungal cells to be transfected,
   an amount of transfection facilitating agent,
(c) incubating the mixture obtained in (b),
(d) analysis of the fungal cells obtained in (c) for transfected cells;

wherein the volume of the mixture obtained in (b) is at most about 25 μL and wherein the compound to be transfected comprises at least a component of a functional polynucleotide-guided genome editing system, preferably a guide-polynucleotide, a polynucleotide encoding a guide-polynucleotide, a polynucleotide-guided genome editing enzyme and/or a polynucleotide encoding a polynucleotide-guided genome editing enzyme.

Further provided is a method for the production of a fungal cell of interest, comprising performing a method for transfection according to the invention and selecting and/or isolating a transfected fungal cell of interest.

Further provided is the use of a transfected fungal cell obtained according to a method according to the invention for the production of a compound of interest comprising culturing the fungal cell under conditions conducive to the production of the compound of interest and optionally purifying and/or isolating the compound of interest.

Further provided is a method for the production of a compound of interest, comprising performing a method for transfection according to the invention and selecting and/or isolating a transfected fungal cell of interest, culturing said fungal cell under conditions conducive to the production of the compound of interest and optionally purifying and/or isolating the compound of interest.

### BRIEF DESCRIPTION OF THE DRAWINGS

FIG. 1 represents plasmid pAMA-hygB depicting the A. niger hygromycin resistance cassette, the AMA1 replication element and the E. coli chloramphenicol selection marker; its sequence is set forward as SEQ ID NO: 3.

### DESCRIPTION OF THE SEQUENCES

SEQ ID NO: 1 sets out the sequence of the protospacer used to target the CRISPR-Cas9 complex to fwnA (An09g05730).

SEQ ID NO: 2 sets out the sequence of the DNA fragment consisting of two 50 bp parts homologous to the sequences flanking the fwnA (An09g05730) open reading frame.

SEQ ID NO: 3 sets out the sequence of pAMA-hygB.

### DETAILED DESCRIPTION OF THE INVENTION

In a first aspect, there is provided for a method for transfecting a compound into fungal cells, preferably filamentous fungal cells, comprising:
(a) providing a container,
(b) dispensing into the container at least:
   an amount of an aqueous solution comprising at least the compound to be transfected,
   an amount of fungal cells to be transfected,
   an amount of transfection facilitating agent,
(c) incubating the mixture obtained in (b),
(d) analysis of the fungal cells obtained in (c) for transfected cells;

wherein the volume of the mixture obtained in (b) is at most about 25 μL and wherein the compound to be transfected comprises at least a component of a functional polynucleotide-guided genome editing system, preferably a guide-polynucleotide, a polynucleotide-guided genome editing enzyme and/or a polynucleotide encoding a polynucleotide-guided genome editing enzyme.

In the embodiments of the invention, transfection is to be construed as the process of bringing a foreign compound into a cell. The term transformation is to be construed as the process of transforming the genotype and/or phenotype of the cell by the transfected compound(s).

In the embodiments of the invention, transfection does preferably not involve micro-injection of cells with nucleic acid, agrobacterium mediated transfection and/or bombardment of cells with nucleic acid coated particles.

The method is herein referred to as a method according to the invention. Specifically preferred embodiments of the invention are set forward in the examples herein. The order of the steps in (b), may be any order.

In the embodiments of the invention, for editing a genome, a donor polynucleotide such as a donor DNA might be required, especially when relying on homologous recombination for genome editing at a desired spot in genomic DNA using a genome editing system. Such donor polynucleotide is known to the person skilled in the art; the person skilled in the art knows how to design a donor polynucleotide (see e.g. Knott G J and Doudna, 2018; Adiego-Pérez et al, 2019; Cai et al, 2019; WU et al, 2018 and the examples herein).

In the embodiments of the invention, the compound to be transfected may be any compound or composition of compounds, such as DNA, RNA and/or protein, but should at least comprise at least a component of a functional polynucleotide-guided genome editing system, preferably a guide-polynucleotide, a polynucleotide encoding a guide-polynucleotide, a polynucleotide-guided genome editing enzyme and/or a polynucleotide encoding a polynucleotide-guided genome editing enzyme. A functional polynucleotide-guided genome editing system is known to the person skilled in the art. Such system may be a CRISPR-Cas9-based system, but other systems are known to the person skilled in the art as well, such as, but not limited to, CRISPR-Cas12a (Cpf1), CRISPR-Cas13a (C2c2) and systems comprising engineered CRISPR-associated nucleases (Knott G J and Doudna, 2018; Adiego-Pérez et al, 2019; Cai et al, 2019; WU et al, 2018). A guide-polynucleotide is known to the person skilled in the art and may e.g. be a guide-RNA or a functional part thereof, a polynucleotide-guided genome editing enzyme is known to the person skilled in the art and may be Cas9, Cas12a, Cas13a or the like. The compound may also be a polynucleotide encoding a guide-polynucleotide and/or a polynucleotide-guided genome editing enzyme as defined here above. The container may be any type of container suitable to perform the method according to the invention and may be a single container or an array of containers (wells) such as a micro-titre-plate (MTP). Such MTP are known to the person skilled in the art and may e.g. comprise 6, 24, 48, 96, 192, 384, 768, 1536, or 3072 wells.

By introduction of a functional polynucleotide-guided genome editing system the present invention unexpectedly leads to a surprising increase in transformation frequency, as number of transformants per microgram of DNA. This increase is from two to fifty fold, or from three to twenty fold or from five to ten fold. Similarly unforeseen, also editing frequency improves substantially with increases from two to fifty fold, or from three to twenty fold or from five to ten fold. This demonstrates a marked improvement in transformation and/or editing frequencies of the method according to the invention compared to a prior art protocol such as described in WO 2008/000715.

The amount of an aqueous solution comprising at least the compound to be transfected may be any amount that works within the method according to the invention. Preferably, the amount is such that the volume of the mixture obtained in step (b) is at most about 20 μL, at most about 15 μL, at most about 10 μL, at most about 5 μL, at most about 4 μL, at most about 3 μL, at most about 2 μL, at most about 1 μL, at most about 500 nL, at most about 200 nL, at most about 100 nL, at most about 50 nL, at most about 25 nL, at most about 10 nL, at most about 5 nL, at most about 4 nL, at most about 3 nL, or at most about 2.5 nL. The amount is preferably at most about 100 nL, at most about 200 nL, at most about 300 nL, at most about 400 nL, at most about 500 nL, at most about 600 nL, at most about 700 nL, at most about 800 nL, at most about 900 nL or at most about 1000 nL (1 μL), at most about 500 nL, at most about 200 nL, at most about 100 nL, at most about 50 nL, at most about 25 nL, at most about 10 nL, at most about 5 nL, at most about 4 nL, at most about 3 nL, at most about 2 nL, or at most about 1 nL. Preferably, the amount is such that the volume of the mixture obtained in step (b) is at most 20 μL, at most 15 μL, at most 10 μL, at most 5 μL, at most 4 μL, at most 3 μL, at most 2 μL, at most 1 μL, at most 500 nL, at most 200 nL, at most 100 nL, at most 50 nL, at most 25 nL, at most 10 nL, at most 5 nL, at most 4 nL, at most 3 nL, or at most 2.5 nL. The amount is preferably at most 100 nL, at most 200 nL, at most 300 nL, at most 400 nL, at most 500 nL, at most 600 nL, at most 700 nL, at most 800 nL, at most 900 nL, at most 1000 nL, at most 500 nL, at most 200 nL, at most 100 nL, at most 50 nL, at most 25 nL, at most 10 nL, at most 5 nL, at most 4 nL, at most 3 nL, at most 2 nL, or at most about 1 nL.

The amount of fungal cells to be transfected may be any amount that works within the method according to the invention. Preferably, the amount is such that the volume of the mixture obtained in step (b) is at most about 20 μL, at most about 15 μL, at most about 10 μL, at most about 5 μL or at most about 4 μL. Preferably, the amount is such that the volume of the mixture obtained in step (b) is at most 20 μL, at most 15 μL, at most 10 μL, at most 5 μL or at most 4 μL. The volume wherein the amount of fungal cells is comprised may be any suitable volume and may be at most about 200 nL, at most about 400 nL, at most about 600 nL, at most about 800 nL, at most about 1000 nL, at most about 1200 nL, at most about 1400 nL, at most about 1600 nL, at most about 1800 nL or at most about 2000 nL (2 μL). The volume wherein the amount of fungal cells is comprised may be any suitable volume and may be at most 200 nL, at most 400 nL, at most 600 nL, at most 800 nL, at most 1000 nL, at most 1200 nL, at most 1400 nL, at most 1600 nL, at most 1800 nL or at most 2000 nL (2 μL). The concentration of fungal cells may e.g. be about 1*10⁵cells/mL, about 1*10⁶ cells/mL, about 1*10⁷cells/mL, or about 1*10⁸ cells/mL. The concentration of fungal cells may e.g. be 1*10⁵cells/mL, 1*10⁶ cells/mL, 1*10⁷ cells/mL, or 1*10⁵ cells/mL.

The amount of transfection facilitating agent may be any amount that works within the method according to the invention. Preferably, the amount is such that the volume of the mixture obtained in step (b) is at most about 20 μL, at most about 15 μL, at most about 10 μL, at most about 5 μL or at most about 4 μL. Preferably, the amount is such that the volume of the mixture obtained in step (b) is at most 20 μL, at most 15 μL, at most 10 μL, at most 5 μL or at most 4 μL. The amount is preferably at most about 200 nL, at most about 400 nL, at most about 600 nL, at most about 700 nL, at most about 800 nL, at most about 1000 nL, at most about 1200 nL, at most about 1400 nL, at most about 1600 nL or at most about 1800 nL. Preferably, the amount is such that the volume of the mixture obtained in step (b) is at most 20 μL, at most 15 μL, at most 10 μL, at most 5 μL or at most 4 μL. The amount is preferably at most 200 nL, at most 400 nL, at most 600 nL, at most 700 nL, at most 800 nL, at most 1000 nL, at most 1200 nL, at most 1400 nL, at most 1600 nL or at most 1800 nL. In the embodiments of the method according to the invention, the transfection facilitating agent may be any transfection facilitating agent known to the person skilled in the art, such as, but not limited to FuGENE® HD (Roche), Lipofectamine™ or Oligofectamine™ (Invitrogen), TransPass™ D1 (New England Biolabs), LypoVec™ or LipoGen™ (Invivogen).

The transfection facilitating agent preferably is polyethylene glycol (PEG). Polyethylene glycol can exist in various molecular weights and grades, such as PEG-4000, PEG-6000, PEG-8000. Preferably the percentage of PEG in the aqueous solution added in step (b) is at most about 35%, at most about 30%, at most about 25%, at most about 20%, or at most about 10%. Preferably the percentage of PEG in the aqueous solution added in step (b) is at most 35%, at most 30%, at most 25%, at most 20%, or at most 10%. The PEG is preferably PEG4000.

After preparing the mixture in (b), the mixture is incubated preferably at Room Temperature (about 20 degrees Celsius) for preferably about 30 minutes, such as 10, 12, 14, 15, 16, 17, 18, 19, 20, 21, 22, 23, 24, 25, 26, 27, 28, 29, 30, 31, 32, 33, 34, 35, 36, 37, 38, 39 or 40 minutes.

After incubating the mixture, the fungal cells obtained in (c) are analysed for transfected cells in step (d). Such analyses may be performed using any means known by the person skilled in the art, such as cell sorting or by culture using a selectable marker that is indicative of successful transfection and successful transfection and transformation of the fungal cells. There is a wide range of selectable markers known to the person skilled in the art and the person skilled in the art how to select an appropriate marker. Such marker can be an auxotrophic marker, a prototrophic marker, a dominant marker, a recessive marker, an antibiotic resistance marker, a catabolic marker, an enzymatic marker, a fluorescent marker, and a luminescent marker. Examples of these are, but are not limited to: amdS (acetamide/fluoroacetamide), ble (phleomycin), hyg (hygromycinR), nat (nourseotricin R), pyrG (uracil/5FOA), niaD (nitrate/chlorate), sutB (sulphate/selenate), GFP (Green Fluorescent Protein), terbinafine and all its different color variants/proteins. In an embodiment, fluorescently labeled polymers (i.e. DNA, RNA or protein) can be introduced during the method according to the invention. An example of such a method is described in WO05040186. The advantages of such embodiment are e.g. that marker-free transfectants can be obtained and that multiple-colors can be used.

Preferably in the embodiments of the method according to the invention, the volume of the mixture obtained in step (b) is at most about 20 μL, at most about 15 μL, at most about 10 μL, at most about 5 μL or at most about 4 μL. Preferably in the method according to the invention, the volume of the mixture obtained in step (b) is at most 20 μL, at most 15 μL, at most 10 μL, at most 5 μL or at most 4 μL.

In the embodiments of the method according to the invention, the fungal cells may be any fungal cells as defined in the section “General definitions” herein. If the fungal cells according to the invention are filamentous fungal cells, the cells are preferably protoplasts. The person skilled in the art knows how to prepare protoplasts. Suitable procedures for preparation of protoplasts are described in EP 238,023 and Yelton et al. (1984, Proc. Natl. Acad. Sci. USA 81:1470-1474). A method to produce protoplasts can be varied to optimize the number and quality of the protoplasts. The person skilled in the art will know that further fine tuning can be performed for each species of fungal cells to obtain the optimized results, not limited to, but involving variations of inoculum size, inoculum method, pre-cultivation media, pre-cultivation times, pre-cultivation temperatures, mixing conditions, washing buffer composition, dilution ratios, buffer composition during lytic enzyme treatment, the type and/or concentration of lytic enzyme used, the time of incubation with lytic enzyme, the protoplast washing procedures and/or buffers, the concentration of protoplasts and/or DNA and/or transfection reagents during the actual transfection, the physical parameters during the transfection, the procedures following the transfection up to the obtained stably transformed fungal cell. The person skilled in the art will understand that these are all state-of-the-art procedures to optimize during transfection of fungal cells. Protoplasts are typically resuspended in an osmotic stabilizing buffer after the actual transfection. The composition of such buffers can vary depending on the species of fungal cells used. Typically these buffers contain an organic component like sucrose, citrate, mannitol or sorbitol between 0.5 and 2 M. More preferably between 0.75 and 1.5 M; most preferred is 1 M. Otherwise these buffers may contain an inorganic osmotic stabilizing component like KCl, MgSO₄, NaCl or MgCl₂ in concentrations between 0.1 and 1.5 M. Preferably between 0.2 and 0.8 M; more preferably between 0.3 and 0.6 M, most preferably 0.4 M. The most preferred stabilizing buffers are STC (sorbitol, 0.8 M; CaCl₂, 25 mM; Tris, 25 mM; pH 8.0) or KCl-citrate (KCl, 0.3-0.6 M; citrate, 0.2% (w/v)). To increase the efficiency of transfection, carrier DNA (such as salmon sperm DNA or non-coding vector DNA) may be added to the transfection mixture.

In the embodiments of the method according to the invention, the incubation of step (c) may be interrupted at least once, wherein an additional amount of an aqueous solution of polyethylene glycol (PEG) is filled into each of the containers. Preferably, the additional amount is such that the volume of the mixture obtained after addition of the additional amount PEG is at most about 20 μL, at most about 15 μL, at most about 10 μL, at most about 5 μL or at most about 4μL. The amount of additional PEG added is preferably at most about 400 nL, at most about 500 nL, at most about 750 nL, at most about 1000 nL, at most about 1500 nL, at most about 2500 nL, at most about 4500 nL, at most about 6500 nL, at most about 7500 nL or at most about 10000 nL. Preferably, the additional amount is such that the volume of the mixture obtained after addition of the additional amount PEG is at most 20 μL, at most 15 μL, at most 10 μL, at most 5 μL or at most 4 μL. The amount of additional PEG added is preferably at most 400 nL, at most 500 nL, at most 750 nL, at most 1000 nL, at most 1500 nL, at most 2500 nL, at most 4500 nL, at most 6500 nL, at most 7500 nL or at most 10000 nL.

In the embodiments of the method according to the invention, the percentage of PEG in the aqueous solution additionally added is at most about 30%, at most about 25%, at most about 20%, or at most about 10%. Preferably the percentage of PEG in the aqueous solution additionally added is at most 35%, at most 30%, at most 25%, at most 20%, or at most 10%. The PEG is preferably PEG4000.

In the embodiments of the method according to the invention, two or more of:
an amount of an aqueous solution comprising at least the compound to be transfected,
an amount of fungal cells to be transfected,
an amount of transfection facilitating agent,

may be combined before being dispensed into the container.

In the embodiment of the method according to the invention, dispensing of the liquids may be performed by any means known to the person skilled in the art, such as by using a pipette, an automated pipet, or a robot device for pipetting, such as a liquids handling device. Preferably, in the embodiments of the method according to the invention, at least one of the dispensing steps in step (b) is performed using non-contact liquid transfer, such as by a liquid handling device. Non-contact liquid transfer and a liquid handling device are known to the person skilled in the art, such as acoustic liquid handling devices, e.g. the Labcyte ECHO 525 acoustic liquid handler.

In the embodiments of the method according to the invention, the analysis in step (d) may comprise culturing the fungal cells in a container containing a volume of culture medium of less than about 2 mL per container. Preferably, an array of containers is used, such as an MTP as defined previously herein. Analysis may involve screening of the transfectants obtained for altered production levels, altered morphology, altered growth rate, altered side product levels, altered color, altered resistance, and the like.

In the embodiments of the method according to the invention, the analysis in step (d) may comprise sorting fungal cells obtained in (c) for transfected cells. Sorting cells, using a flow cytometer, is known to the person skilled in the art. Cells are sorted (identified and isolated) based on a phenotypical feature, such as, but not limited to, expression of a marker.

In a second aspect, there is provided for a method for the production of a fungal cell of interest, comprising performing a method according to first aspect of the invention and selecting and/or isolating a transfected fungal cell of interest. The features of the second aspect of the invention are preferably those of the first aspect of the invention.

In a third aspect, there is provided a transfected fungal cell obtained according to the method of the first or second aspect of the invention for the production of a compound of interest comprising culturing the fungal cell under conditions conducive to the production of the compound of interest and optionally purifying and/or isolating the compound of interest. The features of the third aspect of the invention are preferably those of the first and/or second aspect of the invention.

In a fourth aspect, there is provided a method for the production of a compound of interest, comprising performing a method according to the first aspect of the invention and selecting and/or isolating a transfected fungal cell of interest, culturing said fungal cell under conditions conducive to the production of the compound of interest and optionally purifying and/or isolating the compound of interest. The features of the fourth aspect of the invention are preferably those of the first, second and/or third aspect of the invention.

### Embodiments

The following embodiments of the invention are provided; the features in these embodiments are preferably those as defined previously herein.
1. Method for transfecting a compound into fungal cells, preferably filamentous fungal cells, comprising:
   (a) providing a container,
   (b) dispensing into the container at least:
      an amount of an aqueous solution comprising at least the compound to be transfected,
      an amount of fungal cells to be transfected,
      an amount of transfection facilitating agent,
   (c) incubating the mixture obtained in (b),
   (d) analysis of the fungal cells obtained in (c) for transfected cells;

wherein the volume of the mixture obtained in (b) is at most about 25 μL and wherein the compound to be transfected comprises at least a component of a functional polynucleotide-guided genome editing system, preferably a guide-polynucleotide, a polynucleotide encoding a guide-polynucleotide, a polynucleotide-guided genome editing enzyme and/or a polynucleotide encoding a polynucleotide-guided genome editing enzyme.
2. A method according to embodiment 1, wherein the volume of the mixture obtained in step (b) is at most about 20 μL, at most about 15 μL, at most about 10 μL, at most about 5 μL or at most about 4 μL.
3. A method according to embodiment 1 or 2, wherein the fungal cells in step (b) are protoplasts.
4. A method according to any one of embodiments 1 to 3, wherein the transfection facilitating agent in step (b) is polyethylene glycol.
5. A method according to any one of embodiments 1 to 4, wherein the compound to be transfected in step (b) is DNA, RNA and/or protein.
6. A method according to any one of embodiments 1 to 5, wherein the mixture obtained in step (b) does not contain more than 35% of polyethylene glycol.
7. A method according to any one of embodiments 4 to 6, wherein the incubation of step (c) is interrupted at least once, wherein an additional amount of an aqueous solution of polyethylene glycol is filled into each of the containers.
8. A method according to embodiment 7, wherein the additional amount of aqueous solution does not contain more than 30% polyethylene glycol.
9. A method according to any one of embodiments 1 to 8, wherein two or more of:
   an amount of an aqueous solution comprising at least the compound to be transfected,
   an amount of fungal cells to be transfected,
   an amount of transfection facilitating agent,

are combined before being dispensed into the container.
10. A method according to any one of embodiments 1 to 9, wherein at least one of the dispensing steps in step (b) is performed using non-contact liquid transfer, such as by a liquid handling device.
11. A method according to any one of embodiments 1 to 10, wherein step (d) comprises culturing the fungal cells in a container containing a volume of culture medium of less than about 2 mL per container.
12. A method according to any one of embodiments 1 to 11, wherein step (d) comprises sorting fungal cells obtained in (c) for transfected cells.
13. A method for the production of a fungal cell of interest, comprising performing a method according to any one of embodiments 1 to 12 and selecting and/or isolating a transfected fungal cell of interest.
14. Use of a transfected fungal cell obtained according to the method of any one of embodiments 1 to 13 for the production of a compound of interest comprising culturing the fungal cell under conditions conducive to the production of the compound of interest and optionally purifying and/or isolating the compound of interest.
15. A method for the production of a compound of interest, comprising performing a method according to any one of embodiments 1 to 12 and selecting and/or isolating a transfected fungal cell of interest, culturing said fungal cell under conditions conducive to the production of the compound of interest and optionally purifying and/or isolating the compound of interest.

### General Definitions

Throughout the specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element, two elements or more than two elements.

The word “about” or “approximately” when used in association with a numerical value (e.g. about 10) preferably means that the value may be the given value (of 10) more or less 1% of the value.

Cas9, the single protein component in the class 2 type II-a CRISPR-Cas system (Mohanraju et al., 2016), is capable of complexing with two small RNAs named CRISPR RNA (crRNA) and trans-activating crRNA (tracrRNA) to form a sequence-specific RNA-guided endonuclease (RGEN) whose target specificity is readily reprogrammed by either modifying the crRNA or using a single-chain guide RNA (sgRNA) composed of essential portions of crRNA and tracrRNA (Jinek et al., 2012). Cas9 RGENs cleave chromosomal DNA to produce site-specific DNA double-strand blunt-end breaks (DSBs) that are repaired by homologous recombination (HR) or non-homologous end-joining (NHEJ) to yield genetic modifications (Sander and Joung, 2014).

Cas 12a (Cpf1) is a class 2 type V-a CRISPR RNA guided nuclease (Zetsche et al., 2015; Mohanraju et al., 2016). Cas12a is different compared to Cas9 in various ways. Cpf1 is a single-RNA-guided nuclease and does not require a transactivating CRISPR RNA (tracrRNA), thus gRNAs are shorter in length than those for Cas9 by about 50%. Cas12a cleavage produces cohesive (not blunt) double-stranded DNA breaks leaving 4-5-nt overhanging “sticky” ends, which might facilitate NHEJ-mediated transgene knock-in at target sites. Cas12a recognizes thymidine-rich DNA PAM sequences, for example, 5′-TTTN-3′ or 5′-TTN-3′, which are located at the 5′ end of target-sequences (Zetsche et al., 2015) while Cas9 recognizes guanine-rich (NGG) PAMs located at the 3′-end of the target-sequence (Jinek et al., 2012).

Cas 12a is found in various bacteria including Francisella, Acidaminococcus and Lachnospiraceae (Zetsche et al., 2015). Heterologous Cas12a RGEN activity was demonstrated in mammalian cells (Zetsche et al., 2015; Kim D. et al., 2015), mice (Kim, Y. et al., 2016, Hur et al., 2016), Drosophila (Port and Bullock, 2016) and rice plant (Xu et al., 2016).

A preferred nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O—, S—, or N-alkyl; O—, S—, or N-alkenyl; O—, S— or N-alkynyl; O—, S—, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Herein, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. Preferably, identity and similarity are calculated over the entire length of the sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide according to the invention is represented by a nucleotide sequence. A polypeptide according to the invention is represented by an amino acid sequence. A polynucleotide construct according to the invention is defined as a polynucleotide which is isolated from a naturally occurring gene or which has been modified to contain segments of polynucleotides which are combined or juxtaposed in a manner which would not otherwise exist in nature. Optionally, a polynucleotide present in a polynucleotide construct according to the invention is operably linked to one or more control sequences, which direct the production or expression of the encoded product in a host cell or in a cell-free system.

The sequence information as provided herein should not be so narrowly construed as to require inclusion of erroneously identified bases. The skilled person is capable of identifying such erroneously identified bases and knows how to correct for such errors.

In all embodiments of the invention, the fungal cell according to the invention may be a haploid, diploid or polyploid cell.

A fungal cell according to the invention is interchangeably herein referred as “a cell”, “a cell according to the invention”, “a host cell”, and as “a host cell according to the invention”; said cell may be any fungal cell, i.e. a yeast cell or a filamentous fungus cell. Preferably, the cell is deficient in an NHEJ (non-homologous end joining) component. Said component associated with NHEJ is preferably a yeast Ku70, Ku80, MRE11, RAD50, RAD51, RAD52, XRS2, SIR4, LIF1, NEJ1 and/or LIG4 or homologue thereof. Alternatively, in the cell according to the invention NHEJ may be rendered deficient by use of a compound that inhibits RNA ligase IV, such as SCR7 (Vartak SV and Raghavan, 2015). The person skilled in the art knows how to modulate NHEJ and its effect on RNA-guided nuclease systems, see e.g. WO2014130955A1; Chu et al., 2015; et al., 2015; Song et al., 2015 and Yu et al., 2015; all are herein incorporated by reference. The term “deficiency” is defined elsewhere herein.

When the cell according to the invention is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica ATCC18943, Yarrowia lipolytica CLIB122, Pichia stipidis and Pichia pastoris.

The host cell according to the invention may be a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51. 2011). The filamentous fungal host cell may preferably be a cell of any filamentous form of any of the three families Aspergillaceae, Thermoascaceae and Trichocomaceae, which are accommodated in the taxon Trichocomaceae.

Filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255(ATCC28089); even more preferably the filamentous fungal host cell is an Aspergillus niger. When the host cell is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

Preferably, and more preferably when the microbial host cell is a filamentous fungal host cell, a host cell further comprises one or more modifications in its genome such that the host cell is deficient in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE if compared to a parent host cell and measured under the same conditions.

A modification, preferably in the genome, is construed herein as one or more modifications. A modification, preferably in the genome, of a host cell according to the invention, can either be effected by
a) subjecting a parent host cell to recombinant genetic manipulation techniques; and/or
b) subjecting a parent host cell to (classical) mutagenesis; and/or
c) subjecting a parent host cell to an inhibiting compound or composition.

Modification of a genome of a host cell is herein defined as any event resulting in a change in a polynucleotide sequence in the genome of the host cell.

Preferably, a host cell has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In an embodiment, the mutant host cell comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, (7) electronic access http://nar.oupjournals.org/cgi/reprint/32/7/e59 or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene introduction or gene replacement, partial or complete gene deletion and gene modification.

For example, in case of replacement of a polynucleotide, polynucleotide construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example, a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified.

Alternatively or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K., Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

A modification resulting in reduced or no production of undesired compound is preferably due to a reduced production of the mRNA encoding said undesired compound if compared with a parent microbial host cell which has not been modified and when measured under the same conditions.

A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in WO2008/053019, WO2005/05672A1, WO2005/026356A1, Oliveira et al.; Crook et al., 2014; and/or Barnes et al., may be used at this purpose.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath_et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment, the modification in the genome of the host cell is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell produces 1% less of the undesired compound if compared with the parent host cell which has not been modified and measured under the same conditions, at least 5% less of the undesired compound, at least 10% less of the undesired compound, at least 20% less of the undesired compound, at least 30% less of the undesired compound, at least 40% less of the undesired compound, at least 50% less of the undesired compound, at least 60% less of the undesired compound, at least 70% less of the undesired compound, at least 80% less of the undesired compound, at least 90% less of the undesired compound, at least 91% less of the undesired compound, at least 92% less of the undesired compound, at least 93% less of the undesired compound, at least 94% less of the undesired compound, at least 95% less of the undesired compound, at least 96% less of the undesired compound, at least 97% less of the undesired compound, at least 98% less of the undesired compound, at least 99% less of the undesired compound, at least 99.9% less of the undesired compound, or most preferably 100% less of the undesired compound.

A reference herein to a patent document or other matter which is given as prior art is not to be taken as an admission that that document or matter was known or that the information it contains was part of the common general knowledge as at the priority date of any of the claims.

The sequence information as provided herein should not be so narrowly construed as to require inclusion of erroneously identified bases. The skilled person is capable of identifying such erroneously identified bases and knows how to correct for such errors.

The disclosure of each reference set forth herein is incorporated herein by reference in its entirety.

The invention is further illustrated by the following examples.

### EXAMPLES

In the following Examples, various embodiments of the invention are illustrated. From the above description and these Examples, one skilled in the art can make various changes and modifications of the disclosure to adapt it to various usages and conditions.

### Materials and Methods

### Strains

WT1: Aspergillus niger CBS 513.88 was used as wild-type strain. This strain is available at the Fungal Genetics Stock Center (Manhattan, Kans., USA) under the access number A1513 (McCluskey et al, J. Biosci. (2010) 35(1): 119-126). Aspergillus niger CBS 513.88 was deposited by Gist Brocades (now DSM) with the Centraal Bureau voor Schimmelcultures (Utrecht, the Netherlands) on 10 Aug. 1988, an institute which has been renamed as Westerdijk Fungal Biodiversity Institute. A. niger CBS 513.88 is derived from A. niger NRRL3122 which was deposited in 1964 by a researcher from the Fermentation Research Institute in Ministry of International Trade and Industry located in Chiba, Japan. A. niger NRRL3122 was acquired from the Culture Collection Unit of the Northern Utilization Research and Development Division, US Department of Agriculture, Peoria, Ill., USA. The strain NRRL3122 and its classical derivatives have been in use to produce glucoamylase and acid amylase by Wallerstein Laboratories (USA) since the 1960's. Wallerstein division was part of Baxter-Travenol Laboratories (USA) but was divested in 1977 to Gist-Brocades (the Netherlands), now part of DSM (van Dijck et al Regulatory Toxicology and Pharmacology (2003) 38: 27-35).
GBA 306: The construction of GBA 306 from WT1 has been described in detail in WO2011/009700. The GBA 306 strain has the following genotype: ΔglaA, ΔpepA, ΔhdfA, an adapted BamHI amplicon, ΔamyBII, ΔamyBI, and ΔamyA.

### Molecular Biology Techniques

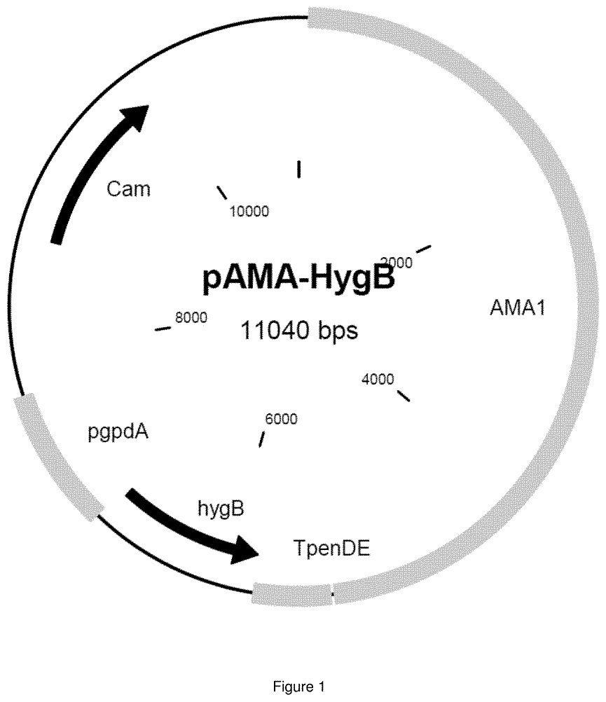

In the strains described here above, using molecular biology techniques known to the skilled person (see: Sambrook & Russell, Molecular Cloning: A Laboratory Manual, 3rd Ed., CSHL Press, Cold Spring Harbor, N.Y., 2001), several genes were over-expressed, and others were down regulated. Examples of the general design of expression vectors for gene over-expression and disruption vectors for down-regulation, transformation, use of markers and selective media can be found in WO199846772, WO199932617, WO2001121779, WO2005095624, WO2006040312, EP635574B, WO2005100573, WO2011009700 and WO2012001169.
Nucleotide and amino acid sequences for A. niger and many other fungal genes, their genomic context and proteins encoded can be derived for example from NCBI (www.ncbi.nlm.nih.gov/) or EMBL (www.ebi.ac.uk.embl/) or AspGD (www.aspergillusgenome.org/). pAMA-hygB plasmids (FIG. 1) can be constructed from AMA expression vectors (as also described and used in for example WO199932617, WO2016110453) by using/inserting a functional hygromycin expression cassette.

TABLE 1
Prior art transfection methods.

| | Van den Bera | Kuivanen et al. |
|---|---|---|
| | (WO2008000715A1) | 2019 |
| Number of Protoplasts | 1.6*10⁵ | ≥1*10⁵ |
| DNA added | 2.5-5.0 μg | 1 μg |
| MTP format | 96 | 96 |
| PEG concentration added | 20%-30% | 25% |
| Total volume after (second) | 130 μL | 87 μL |
| PEG addition | | |
| Total volume after Sorbitol | 380 μL | 200 μL |
| addition | | |
| Subsequent steps: | centrifugation, | plating without top- |
| | washing, plating | agar |

TABLE 2
Comparison of features of a method according to
the invention and prior art transfection methods.

| | Van den Berg | | Kuivanen et al. | | Method according | |
|---|---|---|---|---|---|---|
| | (WO2008000715A1) | | 2019 | | to the invention | |
| Relevant strain genotype | HdfA⁺ | | HdfA⁺ | | HdfA⁻ | |
| Method of selection | Integrative plasmid | | PyrG integration at | | AMA-Hyg plasmid | |
| | with amdS | | genome | | expression | |
| Gene editing method | Homologous | | Cas9 mediated HR | | Cas9 mediated HR | |
| | Recombination (HR) | | | | | |
| Flanking regions | 1000bp | | 500-1000bp | | 50bp | |
| Number of Protoplasts | 1.6*10⁵ | | ≥1*10⁵ | | ≥1.8*10⁴ | |
| DNA added | 2.5-5.0 | μg | 1 | μg | 0.1 | μg |
| Volume of DNA | 5 | μL | 1 | μL | 0.1-0.7 | μL |
| MTP format | 96 | | 96 | | 96-1536 | |
| PEG concentration added | 20%-30% | | 25% | | 20%-30% | |
| Total volume after (second) | 130 | μL | 87 | μL | 10 | μL |
| PEG addition | | | | | | |
| Total volume after Sorbitol | 380 | μL | 200 | μL | 20-100 | μL |
| addition | | | | | | |

| | | | |
|---|---|---|---|
| Subsequent steps: | centrifugation, | plating wo top- | Plating wo top- |
| | washing, plating | agar | agar |

### Example 1

### Low Volume Transfection of Aspergillus niger

Aspergillus niger protoplasts from strain GBA 306 were prepared according to the protocol for transformation in WO199932617 and WO199846772, to obtain a protoplasts suspension of 1*10⁷ protoplasts/mL in STC (1.2 M sorbitol, 10 mM Tris-HCl pH 7.5, 50 mM CaCl2). Transfection of the protoplasts was performed using two different protocols for which detailed descriptions are outlined in Table 3 and Table 4, respectively. Correct transfection resulted in uptake, expression and replication of the plasmid (pAMA-HygB, FIG. 1) containing the hygromycin resistance expression. Transfection as described in Table 3 is based on the method described in WO2008000715A1. For the transfection method of the invention, as depicted in Table 4, 96-well MTP PCR plates and 384-well MTP source plates (Labcyte, San Jose, Calif.) were used. The Labcyte ECHO 525 acoustic liquid handler was used to perform the transfection procedure in MTP according the method of the invention, by adding liquids in an order as listed in Table 4 up until the addition of the 30% PEG solution. Subsequent steps were done using (multi-channel) pipettes. The transfected protoplasts were plated on selective regeneration medium plates (SRM) (see WO199932617 and WO199846772), supplemented with 50 μg/mL hygromycin and grown for 6-7 days at 30° C.

TABLE 3
Protocol for prior art Aspergillus niger transfection in MTP.

| Step | Description | Volume/Remarks |
|---|---|---|
| 1 | 96 well MTP | 1 mL deep-well |
| | | plate |
| 2 | +1.5 μg pAMA-HygB DNA | 10 μL |
| 3 | +20% PEG | 15 μL |
| 4 | + Protoplasts (1*10⁷cells/mL) | 25 μL |
| 5 | 30 min. Room Temperature | |
| 6 | +30% PEG | 90 μL |
| | Total volume | 140 μL |
| 7 | 30 min. Room Temperature | |
| 8 | + Sorbitol 1.2M | 700 μL |
| | Total volume (including sorbitol) | 840 μL |
| 9 | MTP centrifuge | Spin 5 min. 2750 rpm |
| 10 | Aspirate ~760 μL liquid | |
| 11 | Resuspend and transfer transfected | 80 μL |
| | protoplasts to plates with SRM agar | |
| | with hygromycin | |
| 12 | Grow 5-7 days at 30° C. | |

TABLE 4
Protocol for low volume transfection of Aspergillus niger
according to the invention.

| Step | Description | Volume/Remarks | |
|---|---|---|---|
| 1 | 96 well MTP plate | 200 μL 96 well | |
| | | PCR plate | |
| 2 | +0.15 μg pAMA-HygB DNA | 700 | nL |
| 3 | +20% PEG | 1075 | nL |
| 4 | + Protoplasts (1*10⁷cells/mL) | 1800 | nL |
| 5 | 30 min. Room Temperature | | |
| 6 | +30% PEG | 6425 | nL |
| | Total volume | 10 | μL |
| 7 | 30 min. Room Temperature | | |
| 9 | + Sorbitol 1.2M | 90 | μL |
| | Total volume (including sorbitol) | 100 | μL |
| 10 | Transfer transfected protoplasts to | 100 | μL |
| | plates with SRM agar with hygromycin | | |

| | |
|---|---|
| 11 | Grow 5-7 days at 30° C. |

TABLE 5
Comparison of transformation efficiencies between prior art transfection
in MTP and low volume transfection according to the invention.

| | Prior art MTP | Protocol according | Fold |
|---|---|---|---|
| | protocol | to the invention | change |
| Amount of DNA | 1.5 μg | 0.15 μg | 10% |
| Number of protoplasts | 2.5*10⁵ | 1.8*10⁴ | 7% |
| Number of Hyg^{R} transformants | 176 ± 8 | 84 ± 5 | n.a. |
| (average of two plates) | | | |
| Transformation frequency in % | 0.07 | 0.47 | 670% |
| (Transformants/No. Protoplasts added) | | | |
| Transformation frequency | 117 | 560 | 480% |
| (Transformants/μgDNA added) | | | |
| Transformation frequency | 1.3 | 8.4 | 650% |
| (Transformants/μL | | | |
| total volume after addition of PEG) | | | |

Despite the almost fourteen-fold reduction in the number of protoplasts used, the plates obtained by transfection according to the invention showed only a 50% reduction in number of transformed colonies compared to the 96 well 1 mL MTP protocol (Table 5). This corresponds to more than six-fold increase in transformation frequency (transformants per number of protoplasts added) when the transformation was done in low volume instead of the 1 mL MTP protocol. In addition, the transformation frequency, as number of transformants per microgram of DNA, surprisingly increased almost five-fold, by transfection according to the invention compared to the MTP protocol, while tenfold less DNA was used (Table 5). This result shows the clear improvement in transformation frequency of the method according to the invention compared to a prior art protocol.

### Example 2

### Comparison of the Transfection Method According to the Invention to a Prior Art Transfection Method

Aspergillus niger protoplasts from strain GBA 306 were prepared according to the protocol for transformation in WO199932617 and WO199846772, to obtain a protoplasts suspension of 1*10⁷ protoplasts/mL in STC (1.2 M sorbitol, 10 mM Tris-HCl pH 7.5, 50 mM CaCl2). Transfections of the protoplasts was done in a similar way as described in Example 1 (Tables 3 & 4) except for the incubation times in PEG solution. After addition of 20% PEG incubation steps 5 and 7 (Table 3 & 4) are performed for 5 minutes at 4° C. and for 20 minutes at room temperature, respectively. Correct transfection of protoplast results in uptake, expression and replication of the plasmid that was added as circular DNA (pAMA-HygB, FIG. 1) containing the hygromycin resistance expression cassette to the volume in micro titer plates (MTP). The transfected protoplasts were plated on selective regeneration medium plates (SRM) (see WO199932617 and WO199846772), supplemented with 50 μg/mL hygromycin and grown for 6-7 days at 30° C.

TABLE 6
Comparison of transformation efficiencies between prior art transfection
in MTP and low volume transfection according to the invention.

| | MTP protocol | Protocol according | Fold |
|---|---|---|---|
| | Kuivanen et al. | to the invention | change |
| Amount of DNA | 1.5 μg | 0.15 μg | 10% |
| Number of protoplasts | 2.5*10⁵ | 1.8*10⁴ | 7% |
| Number of Hyg^{R} transformants | 308 ± 8 | 34 ± 1 | n.a. |
| (average of two plates) | | | |
| Transformation frequency | 0.12 | 0.19 | 158% |
| in %(Transformants/No. Protoplasts added) | | | |
| Transformation frequency | 205 | 227 | 111% |
| (Transformants/μg DNA added) | | | |
| Transformation frequency | 2.2 | 3.4 | 155% |
| (Transformants/μL | | | |
| total volume after addition PEG) | | | |

After incubation, the plates obtained by transfection using the method according to the invention with the acoustic liquid handler compared to the MTP protocol (Kuivanen et al), showed a 58% increase in transformation frequency (transformants per number of protoplasts added) when the transformation was done using the low volume protocol according to the invention, while tenfold less DNA was used (Table 6). Moreover, the transformation frequency, as number of transformants per microgram of DNA, increased by 11%, by transfection using the method according to the invention with the acoustic liquid handler compared to the 96 MTP protocol, while tenfold less DNA was used (Table 6). This result showed the improvement in transformation frequency using the method according to the invention compared to a prior art transformation protocol.

### Example 3

### Transformation of Aspergillus niger Using CRISPR-Cas

Aspergillus niger protoplasts from strain GBA 306 were prepared as described in example 1 (see WO199932617 and WO199846772), to obtain a protoplasts suspension of 1*10⁷ protoplasts/mL in STC. The deletion of fwnA (An09g05730) was mediated by using CRISPR-Cas prepared by combining purified Cas9 protein from S. pyogenes and a crRNA/tracrRNA complex (both supplied by IDT, Coralville, Iowa, USA). The crRNA/tracrRNA complex was made by adding 0.1 volume duplex buffer (1M potassium acetate, 300 mM HEPES, pH 7.5) to 0.1 mM crRNA (containing the protospacer sequence in fwnA, SEQ ID NO: 1) and 0.1 mM tracrRNA and subsequently incubated at 95° C. for 5 min. In addition to the CRISPR-Cas complex, which induces a double strand break within the fwnA locus, pAMA-HygB (FIG. 1) as well as 100 bp donor DNA fragment (SEQ ID NO: 2) homologous to 50 bp upstream and downstream of the fwnA locus was added . Transfection as described in Table 7 was largely based on method described in WO2008000715A1. The low volume transfection method described in Table 8, 96-well MTP PCR plates and 384-well MTP source plates (Labcyte) were used. The Labcyte ECHO 525 acoustic liquid handler was used to perform the transfection procedure, by adding liquids in the order as listed in Table 2 up until the addition of 30% PEG solution. Subsequent steps were performed using (multi-channel) pipettes. The transfected protoplasts were plated on selective regeneration medium plates (SRM) (see WO199932617 and WO199846772) supplemented with 50 μg/mL hygromycin and incubated for 6-7 days at 30° C.

Improved editing frequency, as demonstrated by the correct integration of a linear donor fragment in the genome, mediated by CRISPR-Cas, was shown to be almost five times more efficient in the low volume transfection method according to the invention, compared to the 1 mL MTP protocol (Table 9). In addition, the transformation frequency, as number of transformants per microgram of DNA, surprisingly increased more than two-fold, by transfection using the method according to the invention compared to the prior art MTP protocol, while tenfold less DNA was used (Table 9). This result shows the clear improvement in transformation frequency using a CRISPR-Cas-mediated transformation using the method according to the invention compared to prior art protocols. The method used in this example employed a CRISPR-Cas complex in combination with an AMA vector and donor DNA for targeted gene deletion.

TABLE 7
Protocol for prior art Aspergillus niger
transformation using CRISPR-Cas in MTP

| Step | Description | Volume/Remarks |
|---|---|---|
| 1 | 96 well MTP | 1 mL deep-well |
| | | plate |
| 2 | +1.5 μg Cas9 protein | 1 μL |
| 3 | +0.1 mM crRNA/tracrRNA complex | 2 μL |
| 4 | +2.0 μg Linear donor DNA | 2 μL |
| | (100bp length) | |
| 5 | + MiliQ | 3 μL |
| 6 | +1.5 μg pAMA-HygB DNA | 2 μL |
| 8 | +20% PEG | 15 μL |
| 9 | + Protoplasts (1*10⁷ cells/mL) | 25 μL |
| 10 | 30 min. Room Temperature | |
| 11 | +30% PEG | 90 μL |
| | Total volume | 140 μL |
| 12 | 30 min. Room Temperature | |
| | + Sorbitol 1.2M | 700 μL |
| 13 | Total volume (including sorbitol) | 840 μL |
| 14 | MTP centrifuge | spin 5 min. 2750 rpm |
| 15 | Aspirate ~760 μL liquid | |
| 16 | Resuspend and transfer transfected | 80 μL |
| | protoplasts to plates with SRM agar | |
| | with hygromycin | |
| 17 | Grow 5-7 days at 30° C. | |

TABLE 8
Protocol for low-volume Aspergillus niger transformation
according to the invention using CRISPR-Cas.

| | Step | Volume/Remarks | |
|---|---|---|---|
| 1 | 96 well MTP plate | 200 μL 96 well | |
| | | PCR plate | |
| 2 | +0.1125 μg Cas9 protein | 75 | nL |
| 3 | +0.1 mM crRNA/tracrRNA complex | 150 | nL |
| 4 | +0.3 μg Linear donor DNA | 150 | nL |
| | (100bp length) | | |
| 5 | + MiliQ | 175 | nL |
| 6 | +0.1125 μg pAMA-HygB DNA | 150 | nL |
| 7 | +20% PEG | 1075 | nL |
| 8 | + Protoplasts (1*10⁷ cells/mL) | 1800 | nL |
| 9 | 30 min. Room Temperature | | |
| 10 | +30% PEG | 6425 | nL |
| | Total volume | 10 | μL |
| 11 | 30 min. Room Temperature | | |
| 12 | + Sorbitol 1.2M | 90 | μL |
| 13 | Transfer transfected protoplasts to plates | 100 | μL |
| | with SRM agar with hygromycin | | |
| 14 | Grow 5-7 days at 30° C. | | |

TABLE 9
Comparison of transformation efficiencies using CRISPR-Cas in prior art
MTP transfection and low volume transfection according to the invention.

| | Prior art | Protocol according | Fold |
|---|---|---|---|
| | protocol | to the invention | change |
| Quantity of donor DNA | 2.0 μg | 0.3 μg | 15% |
| Number of protoplasts | 2.5*10⁵ | 1.8*10⁴ | 7% |
| Number of transformants (Hyg^{R}) | 60 ± 10 | 9 ± 1 | n.a. |
| (average of two plates) | | | |
| ΔfwnA (An09g05730) edited transformants | 11 ± 3 | 4 ± 1 | n.a. |
| (average of two plates) | | | |
| Transformation frequency in % | 0.024 | 0.050 | 208% |
| (Transformants/No. Protoplasts added) | | | |
| Transformation frequency | 30 | 30 | 100% |
| (Transformants/μg DNA added) | | | |
| Transformation frequency | 0.43 | 0.90 | 210% |
| (Transformants/μL | | | |
| total volume after addition PEG) | | | |
| Editing frequency (percentage of edited | 18% | 44% | 410% |
| transformants) | | | |
| Editing frequency (percentage edited | 0.004% | 0.022% | 505% |
| transformants/protoplasts) | | | |
| Editing frequency (Number of edited | 5.5 μg⁻¹ | 13 μg⁻¹ | 242% |
| transformants/μg donor DNA added. | | | |
| Editing frequency (Number of edited | 0.08 | 0.40 | 509% |
| transformants/μL total volume | | | |
| after addition PEG) | | | |

### REFERENCES

Knott G J and Doudna. CRISPR-Cas guides the future of genetic engineering. Science. 2018 Aug. 31; 361(6405):866-869.
Belén Adiego-Pérez, Paola Randazzo, Jean Marc Daran, René Verwaal, Johannes A. Roubos, Pascale Daran-Lapujade, John van der Oost. Multiplex genome editing of microorganisms using CRISPR-Cas. 2019 May 14; FEMS Microbiology Letters, 366, 8,
Peng Cai, Jiaoqi Gao and Yongjin Zhou. CRISPR-mediated genome editing in non-conventional yeasts for biotechnological applications. Microbial Cell Factories volume 18, Article number: 63 (2019).
Wen Y. Wu, Joyce H. G. Lebbink, Roland Kanaar, Niels Geijsen and John van der Oost. Genome editing by natural and engineered CRISPR-associated nucleases. Nature Chemical Biology volume 14, pages 642-651 (2018).

1. A method for transfecting a compound into fungal cells, optionally filamentous fungal cells, comprising:
(a) providing a container,
(b) dispensing into the container at least:
an amount of an aqueous solution comprising at least the compound to be transfected,
an amount of fungal cells to be transfected,
an amount of transfection facilitating agent,
(c) incubating the mixture obtained in (b),
(d) analysis of the fungal cells obtained in (c) for transfected cells;
wherein the volume of the mixture obtained in (b) is at most about 25 μL and wherein the compound to be transfected comprises at least a component of a functional polynucleotide-guided genome editing system, optionally a guide-polynucleotide, a polynucleotide encoding a guide-polynucleotide, a polynucleotide-guided genome editing enzyme and/or a polynucleotide encoding a polynucleotide-guided genome editing enzyme.

2. The method according to claim 1, wherein the volume of the mixture obtained in (b) is at most about 20 μL, at most about 15 μL, at most about 10 μL, at most about 5 μL or at most about 4 μL.

3. The method according to claim 1, wherein the fungal cells in (b) are protoplasts.

4. A The method according to claim 1, wherein the transfection facilitating agent in (b) is polyethylene glycol.

5. The method according to claim 1, wherein the compound to be transfected in (b) is DNA, RNA and/or protein.

6. The method according to claim 1, wherein the mixture obtained in (b) does not contain more than 35% of polyethylene glycol.

7. The method according to claim 4, wherein the incubation of (c) is interrupted at least once, wherein an additional amount of an aqueous solution of polyethylene glycol is filled into the container.

8. The method according to claim 7, wherein an additional amount of aqueous solution does not contain more than 35% polyethylene glycol.

9. The method according to claim 1, wherein two or more of:
an amount of an aqueous solution comprising at least the compound to be transfected,
an amount of fungal cells to be transfected,
an amount of transfection facilitating agent,
are combined before being dispensed into the container.

10. The method according to claim 1, wherein at least one item dispensed in (b) is performed using non-contact liquid transfer, optionally by a liquid handling device.

11. The method according to claim 1, wherein (d) comprises culturing the fungal cells in a container containing a volume of culture medium of less than about 2 mL per container.

12. The method according to claim 1, wherein (d) comprises sorting fungal cells obtained in (c) for transfected cells.

13. A method for the production of a fungal cell of interest, comprising performing a method according to claim 1 and selecting and/or isolating a transfected fungal cell of interest.

14. A product comprising a transfected fungal cell obtained according to the method of claim 1 for production of a compound of interest comprising culturing the fungal cell under conditions conducive to the production of the compound of interest and optionally purifying and/or isolating the compound of interest.

15. A method for production of a compound of interest, comprising performing a method according to claim 1 and selecting and/or isolating a transfected fungal cell of interest, culturing said fungal cell under conditions conducive to the production of the compound of interest and optionally purifying and/or isolating the compound of interest.


SEQUENCE LISTING (3 sequences)

SEQ ID NO: 1 - DNA, 20 bp, Artificial Sequence
  Sequence of the protospacer used to target the CRISPR-Cas9 complex to fwnA (An09g05730)
tcagatatattcagtcactg

SEQ ID NO: 2 - DNA, 100 bp, Artificial Sequence
  Sequence of the DNA fragment consisting of two 50bp parts homologous to the sequences flanking the fwnA (An09g05730) open reading frame
ttgcatcgcttgcgttcatgatgaacccagacaggtgtccaaaactgtcagactgtagctgttgctccatcacttcgagtctcaatgaccttctgagccg

SEQ ID NO: 3 - DNA, 11040 bp, Artificial Sequence
  Sequence of pAMA-hygB
ggtaccacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctg